(19) United States
(12) Patent Application Publication
(10) Pub. No.: US 2023/0001149 A1
(43) Pub. Date: Jan. 5, 2023

(54) ANCHORING ELEMENTS FOR A STEERABLE DEVICE

(71) Applicant: BASECAMP VASCULAR, REIMS (FR)

(72) Inventors: Jean-Baptiste CAZENEUVE, Ivry-sur-seine (FR); Camille MAIANO, Brélès (FR)

(73) Assignee: BASECAMP VASCULAR, REIMS (FR)

(21) Appl. No.: 17/782,006

(22) PCT Filed: Dec. 4, 2020
(86) PCT No.: PCT/EP2020/084725

§ 371 Date: Jun. 2, 2022

(30) Foreign Application Priority Data
Dec. 6, 2019 (EP) 19306590.1

(51) Int. Cl. A61M 25/01

(52) U.S. Cl. CPC A61M 25/0147; A61M 2205/0266; A61M 2025/015

(57) ABSTRACT

A steerable device including a flexible axially elongated member, at least one actuating element arranged alongside the periphery of the elongated member, at least one fastening element configured to fasten at least partially the at least one actuating element to the flexible elongated member distal end, the fastening element being in direct contact with the flexible elongated member, at least one anti-return element configured to keep the at least one actuating element from sliding alongside the periphery of the flexible elongated member distal end, wherein the at least one anti-return element and the at least one fastening element are in axial abutting contact.

### FIELD OF INVENTION

The present invention relates to a steerable device, for instance, such device is used as a guide for a catheter or an endoscope for internally investigating a tubular element such as a pipe, a duct or an artery. The device of the invention is particularly suitable for use in the field of surgical investigation inside the body of a subject.

Even though the device is designed for surgical purpose, it can also be used in other areas needing non-destructive control or diagnostic such as pipeline systems for instance.

### BACKGROUND OF INVENTION

There is a wide range of applications where there is a need for using a device inside a pipe, a duct or a tube, for example for the placement of the distal part of a flexible tube in a specific location, for investigation or bringing pharmaceuticals, or functionality at a remote or hard-to-access location.

When displacing a flexible elongated device in the lumen of a pipe, duct or a tube, it is important for the user to be able to control carefully and precisely the movement and the placement of such device. Placement of such devices within pipes is a known technical issue in oil engineering or in motor engineering. Placement of devices within body tubes, such as for example through the ostium (of a vein, an artery, the gastro-intestinal track . . . etc), is also known in the medical field as being challenging.

In the medical field, using surgical or endovascular techniques, one can treat a lot of cardiovascular diseases causing death in the world. One of the pathologies encountered is the myocardial infarction along with peripheral vascular diseases. Through the last decades, the use of catheters and guidewires to reach pathological areas so as to deliver stents or balloons has emerged as an easy-to-implement solution. These endovascular techniques are less invasive compared to conventional surgeries. They come with a decreased recovery time and less post operation complications.

However, generally speaking, the surgeon skill and experience are a major success factor in the complex interventions while recent developments aim at facilitating navigation through complex anatomies as independently as possible from those surgeon skill and experience.

To do so, for example, it is known the application WO95/06494 that discloses a flexible elongated device comprising a flexible elongate member with proximal and distal extremities. A shape memory element disposed in the flexible elongate member and capable of assuming martensitic and austenitic states and having first and second portions. A layer of conductive material formed on at least one of said portions. The layer has a conductivity greater than that of the shape memory element. Electrical current is supplied to the shape memory element. The conductive layer serves to conduct current and shunts current flow around that portion of the shape memory element having the layer of conductive material thereon. This disclosure requires however a complex assembly with multiple inner connections inside the elongated device with a high risk of having a disassembly between the shape memory element and its associated inner knot.

So as to overcome such complexity, lower disassembly risks and improve manufacturability, it is also known the patent application WO2018167300 disclosing a an elongated steerable system for guiding a catheter or an endoscope, comprising: an elongated flexible member comprising, along at least a part of its longitudinal axis, at least one projection which, in transversal section, projects from the elongated flexible member; said at least one projection having a distal end and a proximal end, and defining, in transversal section, two lateral sides projecting from the elongated flexible member; and a wire; wherein said at least one projection comprises at least one transverse retaining and passing means near the distal end of said projection, said retaining and passing means extending transversely to the lateral sides of the projection; and the wire passes through said retaining and passing means alternatively from one lateral side of the projection to the other lateral side of the projection, wherein adherence between the wire and the retaining and passing means retains the wire ensuring the anchorage of the wire to said retaining and passing means.

These prior art devices are either complex and/or cumbersome and do not offer enough bending ability to navigate inside the lumen of a tube or pipe. The main objective of the invention is to provide a steerable device comprising actuators and fastening means that are easy to produce, compact and repeatable. According to the invention, the steerable device obtained is flexible enough to allow easy navigation inside a lumen, tube or pipe.

### SUMMARY

This invention thus relates to a steerable device configured to be advanced in the lumen of a tubular element, said device comprising:
a flexible axially elongated member having proximal and distal ends,
at least one actuating means arranged alongside the periphery of said elongated member,
at least one fastening means configured to fasten at least partially the at least one actuating means to the flexible elongated member distal end, said fastening means being in direct contact with the flexible elongated member,
at least one anti-return means configured to keep the at least one actuating means from sliding alongside the periphery of the flexible elongated member distal end,
   characterized in that the at least one anti-return means and the at least one fastening means are in axial abutting contact so as to prevent the at least one actuating means from sliding once actuated.

In a preferred embodiment, the at least one actuating means is made of a shape memory alloy such as NiTi alloy. Such alloys are used in the invention in surgical domain for their biocompatibility as well as their low density. The actuated bending motion is obtained by implementing electrical current that induces a phase transformation in the material through the Joule effect. Once the current no longer flows, the actuating means goes back to initial straight position thanks to the assembly spring back force. The higher the current, the higher the bending angle until a threshold.

In a preferred embodiment, the at least one actuating means comprises a spring or a wire that are more practical, lighter and more compact for a small diameter pipe, lumen or tube.

Preferably, the at least one flexible axially elongated member is wire or blade shaped. It may have a cross-section profile under a form selected from star, circular, semicircular, square, rectangular, triangle, pyramidal or any combinations thereof.

In another alternative, the at least one flexible axially elongated member is spring shaped. More preferably, the spring is a helicoidal spring.

Preferably, the at least one flexible axially elongated member is blade shaped so as to improve assembly and to have a preferred common bending plane for the actuators. In an alternative embodiment, the flexible axially elongated member is wire shaped.

In an alternative embodiment, the fastening means is a ligation around the flexible elongated member. Just like for the actuating means, ligations are easy to implement and reduce the spatial congestion.

In an alternative embodiment, the anti-return means is a glue filled tube, the actuating means going through said tube. The advantage of this configuration is that the actuating means are very solidly anchored while the tube end thickness serves as axial abutments for the fastening means.

In a variant, the anti-return means is a crimped tube, the actuating means going through said tube. The advantage of this alternative configuration is that the actuating means are also very solidly anchored while the tube end thickness serves as axial abutments for the fastening means.

Advantageously, another second anti-return means is made integral with the flexible elongated member so as to maintain the fastening means in place. Particularly, said anti-return means being a recess built into the flexible elongated member and configured to host the at least one fastening means. Preferably, the recess extends in a direction perpendicular to the longitudinal axis of the flexible elongated member. In this case with the anti-return means made integral with the flexible elongated member, the device according to the invention is easy to produce, compact, and presents a low disassembly risk for the anti-return means. In addition, the assembly robustness is improved.

In an exemplary embodiment, the anti-return means is made integral with the actuating means. In this other configuration also, the device according to the invention is easy to produce, compact, and presents a low disassembly risk for the anti-return means

In an exemplary embodiment, the at least one anti-return means is a loop encapsulating at least partially the at least one fastening means or said loop being an abutment for the at least one fastening means. The loop is obtained by modifying the shape of the actuating means. It is also possible to have a configuration where the at least one anti-return means is a knot, a weld or any local reinforcement located at the at least one actuating means distal end. These configurations add simplicity to the low disassembly risk.

Advantageously, the at least one anti-return means is a groove built into the actuating means and configured to host the at least one fastening means. This configuration adds simplicity to the low disassembly risk. Preferably, the groove extends in a direction perpendicular to the longitudinal axis of the flexible elongated member. The groove can be obtained by an indentation process.

In an advantageous embodiment, the at least one actuating means is inside an electrically isolating material such as a tube or a coating. The material has preferably an electrical and/or temperature isolation effect.

In another advantageous embodiment, the at least one actuating means is arranged alongside the external periphery of the elongated member. In this case, the device assembly is made easier.

In another advantageous embodiment, the at least one actuating means is arranged alongside the internal periphery of the elongated member. In this case, the external diameter is constant and the device according to the invention is less bulky.

The invention has also as an object an assembly method for steerable device according to the invention comprising the steps of:
providing a flexible axially elongated member having proximal and distal ends,
arranging at least one actuating means alongside the periphery of said elongated member,
fastening the at least one actuating means to the flexible elongated member distal end with at least one fastening means,
Providing at least one anti-return means configured to keep the at least one actuating means from sliding alongside the periphery of the flexible elongated member distal end,
   characterized in that the at least one anti-return means and the at least one fastening means are in axial abutting contact so as to prevent the at least one actuating means from sliding once actuated.

In a preferred embodiment, in the method according to the invention, the at least one actuating means is arranged alongside the external periphery of the elongated member. In this case, the device assembly is made easier.

In another advantageous embodiment, in the method according to the invention, the at least one actuating means is arranged alongside the internal periphery of the elongated member. In this case, the external diameter is constant and the device obtained according to the method of the invention is less bulky.

The assembly method according to the invention may be such that the at least one actuating means is a wire, the at least one fastening means is a ligation and the at least one anti-return means is obtained by increasing the contact pressure between the wire and the ligation , with said ligation going alternatively up and under the wire so as to ligate longitudinally partial parts of the wire with the flexible elongated member. This method is simple to implement and reduces spatial congestion for the device according to the invention.

### DEFINITIONS

In the present invention, the following terms have the following meanings:
“About”: is used herein to mean approximately, roughly, around, or in the region of. When the term “about” is used in conjunction with a numerical range, it modifies that range by extending the boundaries above and below the numerical values set forth.

According to one embodiment, the term “about” preceding a figure means plus or less 10% of the value of said figure.
“Actuator”: can be any type of string, cable, wire, ribbon, tube or any set of those, capable of activating the body part to which it is fixed in order to trigger a function or to induce a bending of an area of the body part to which it is fixed. Actuators may be materials and devices that are able to change their shape (shape memory material) in response to changes in environmental conditions, temperature, and perform mechanical work, but this not an exclusive option. An actuator may convey energy. Most of the time, an actuator transforms the received energy into another type of energy. In one embodiment, the actuator receives an electrical current which is transformed into heat thanks to Joule effect, and upon action of the heat, contracts.
“Catheter” is a tubular medical device for insertion into canals, vessels, passageways or body cavities for diagnostic, surgical or therapeutic purposes such as to permit injection/withdrawal of fluids, to keep passageways open, to inspect internal organs and tissues and to place medical tools into position for medical treatment within the body of an animal or of a human. In this invention, the term “catheter” encompasses any cannula or medical probe designed for insertion in a human or animal canal, vessel, passageway or body cavity.
“to curvate”: means to take the form of a curvature, to bend. Having a curvature or being curved is used in opposition to being straight. The term “curvature” means non-zero curvature. The curvature can be positive or negative.
“Elongated”: refers to an element such as a body, device, or a system that extends longitudinally.
“Means”: refers to elements or configurations in the context of the invention. “Flexible”: refers to an object that may bend without breaking.
“Anti-return means”: refers to an element or a configuration that is able to keep another element to which it is connected, from sliding. In the case of a configuration, such configuration keeps the target element from sliding.

### BRIEF DESCRIPTION OF THE DRAWINGS

FIGS. 1A and 1B are illustrations of the device according to the invention with recesses in the flexible axially elongated member as anti-return means in top view and perspective views. The perspective view presents a different configuration for the flat wire actuating means which has smaller width.

FIGS. 2A, 2B and 2C are illustrations of the device according to the invention with a loop as anti-return means, such loop standing alone (2A), encapsulating partially the fastening means (2B) and encapsulating totally the fastening means (2C).

FIGS. 3A and 3B are illustrations of the device according to the invention with a knot as anti-return means, FIG. 3A shows one knot and FIG. 3B, two knots.

FIG. 4 is an illustration of the device according to the invention with the anti-return means being a configuration with the actuating means passing in and out the ligation as fastening means.

FIGS. 5A, 5B and 5C are illustrations of the device according to the invention with weld seams as anti-return means made integral with the actuating means, such weld seams being longitudinally spaced apart and separated by the fastening mean windings (5A), continuous and uniformly distributed along the actuating means (5B) and as a local reinforcement (5C).

FIGS. 6A, 6B and 6C are three different illustrations of the device according to the invention with grooves built into the actuating means as anti-return means, such grooves being longitudinally spaced apart.

FIGS. 7A and 7B are illustrations of the device according to the invention with a tube, crimped (7A) or glue filled (7B), as anti-return means.

FIGS. 8A and 8B are illustrations of the device according to the invention showing an embodiment wherein the anti- return means and the actuating means are arranged alongside the internal periphery of the flexible elongated member. FIG. 8A shows the inner part of the flexible elongated member while FIG. 8B is an view from the outside.

### DETAILED DESCRIPTION

The following detailed description will be better understood when read in conjunction with the drawings. For the purpose of illustrating, the device according to the invention is shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

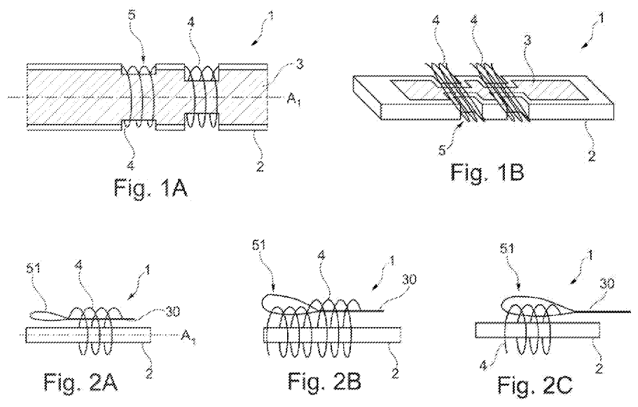

In FIGS. 1A and 1B, one can see an exemplary configuration of the device 1 according to the invention with recesses 5 in the flexible axially elongated member 2 as anti-return means in top and perspective views. In this figure, the flexible axially elongated member 2 is a blade, such blade has been made integral with the anti-return means 5 in the form of two recesses. A blade is used in this embodiment, but a tubular element is also a possibility for the flexible, axially elongated member 2. The anti-return means 5, here the recesses, extend transversally towards the axis A1 of the elongated member 2. The actuating means 3 is a flat wire extending longitudinally while being in facial contact with the elongated member 2. It is thin compared to the ligation 4 diameter. Each of the anti-return means 5 host ligations 4 as wires winding around said anti-return means 5 forming coil windings with their axis being the same as the axis A1 of elongation of the elongated member 2. The ligation 4 ties the actuating means 3 (here, a flat wire) and fastens it to the elongated member 2 thanks to the grooves made in the actuating means 3. The combination of the ligation 4, the actuating mean grooves (1B) and potentially the recesses (generally speaking, any kind of anti-return means 5) allow to keep the actuating mean 3 from sliding when actuated for instance by Joule effect when the actuating means 3 is a shape memory alloy such as NiTi type. In a preferred embodiment, the recess base of the flexible axially elongated member 2 and the groove base of the actuating means 3 barely coincide radially as depicted in FIG. 1A.

Regardless of the embodiment, the device 1 displays a diameter comprised between 200 μm and 5 mm.

In FIGS. 2A, 2B and 2C, one can see an exemplary configuration of the device 1 according to the invention with loops 51 made integral with the wire 30 as anti-return means. In this figure, the flexible axially elongated member 2 is a blade but could have been a tubular element. In FIG. 2A, the loop 51 serves as anti-return means. It is in axial abutment contact with the ligation 4 that ties the wire 30 to the elongated member 2. In FIG. 2B, the loop 51 serves as anti-return means. It is in axial abutment contact with one part of the ligation 4 that ties the wire 30 to the elongated member 2 while also encapsulating i.e encompassing another part of the ligation 4 so as to improve the anti-return effect. In FIG. 2C, the loop 51 serves as anti-return means. It is in axial abutment contact with the ligation 4 that ties the wire 30 to the elongated member 2 while also encapsulating i.e encompassing said ligation 4 so as to improve the anti-return effect. The windings of the ligation 4 are trapped within the close loop 51 made of the wire 30. The combination of the ligation 4 that ties the wire 30 to the elongated member 2 and the close loop 51 keeps the wire 30 from sliding when actuated for instance by Joule effect when the wire 30 is a shape memory alloy such as NiTi type.

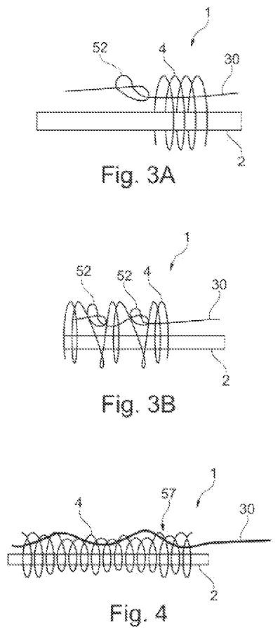

In FIGS. 3A and 3B, one can see an exemplary configuration of the device 1 according to the invention with two knots 52 made integral with the wire 30 as anti-return means. In this figure, the flexible axially elongated member 2 is a blade but could have been a tubular element. In FIG. 3A, there is only one knot and in FIG. 3B, there are two knots spaced apart longitudinally. the knot(s) 52 serve(s) as anti-return means. They are in axial abutment contact with the ligation 4 that ties the wire 30 to the elongated member 2.

In FIG. 4, the anti-return means 57 is a configuration where the wire 30 and the ligation 4 are intertwined (not exactly illustrated for clarity purpose) with the wire 30 going alternatively up and under the windings of the ligation 4 so as to ligate longitudinally partial parts of the wire 30 with the flexible elongated member 2. This configuration increases the contact pressure between the actuating means 30 and the ligation 4, thus the adherence between the two latter is increased which is what creates the axial abutment. The ligation 4 ties the wire 30 to the elongated member 2 to keep the wire 30 from sliding when actuated for instance by Joule effect when the wire 30 is a shape memory alloy such as NiTi type.

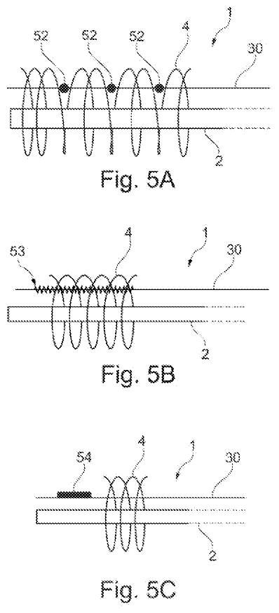

In FIGS. 5A, 5B and 5C, one can see an exemplary configuration of the device 1 according to the invention with added material 52, 53 and 54 made integral with the wire 30 as anti-return means. In this figure, the flexible axially elongated member 2 is a blade but could have been a tubular element. In FIG. 5A, the three weld seams serve as anti-return means. They are spaced apart longitudinally and in axial abutment contact with the ligation 4 windings that tie the wire 30 to the elongated member 2. In FIG. 5B, the discontinuous weld spots 53 serve as anti-return means. They are in axial abutment contact with the ligation 4 windings that tie the wire 30 to the elongated member 2. In FIG. 5C, the local reinforcement 54 serves as anti-return means, such local reinforcement 54 is axially offset in FIG. 5C but could be located in between both ends of the ligation 4. The local reinforcement can be the result of a weld or not. Said local reinforcement 54 is in axial abutment contact with the ligation 4 windings that tie the wire 30 to the elongated member 2. The ligation 4 ties the wire 30 to the elongated member 2. The combination of the ligation and the anti-return means allows to keep the wire 30 from sliding when actuated for instance by Joule effect when the wire 30 is a shape memory alloy such as NiTi type.

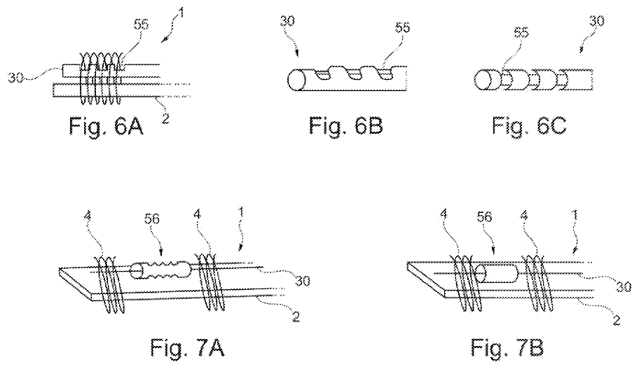

In FIGS. 6A, 6B and 6C, one can see an exemplary configuration of the device 1 according to the invention with grooves 55 made integral with the wire 30 as anti-return means. In this figure, the flexible axially elongated member 2 is a blade but could have been a tubular element. In FIG. 6A, the three grooves serve as anti-return means. They are spaced apart longitudinally and in axial abutment contact with the hosted ligation 4 windings that ties the wire 30 to the elongated member 2. Said grooves can be obtained by different processes such as for example indentation, pressure flattening, or even laser localized fusion. FIGS. 6B and 6C are illustrations of the grooves obtained on the wire with the different methods given as examples. The ligation 4 ties the wire 30 to the elongated member 2. The combination of the ligation and the at least one groove allows to keep the wire 30 from sliding when actuated for instance by Joule effect when the wire 30 is a shape memory alloy such as NiTi type.

FIGS. 7A and 7B are embodiments, where the anti-return means 56 is tube that is either crimped (FIG. 7A) or filled with glue (FIG. 7B). In both cases, the tube 56 comprises the wire 30 that goes through said tube 56. The glue or the crimping serves as a blocking element that holds the wire 30 in place. In FIGS. 7A and 7B, the tube 56 has ligation 4 windings on both sides but a configuration with only one ligation 4 winding is possible as long as an axial abutment contact is obtained. The combination of the ligation 4 tying the wire 30 to the elongated member 2 in addition to the crimping or glue effect allows keeping the wire 30 from sliding when actuated for instance by Joule effect when the wire 30 is a shape memory alloy such as NiTi type.

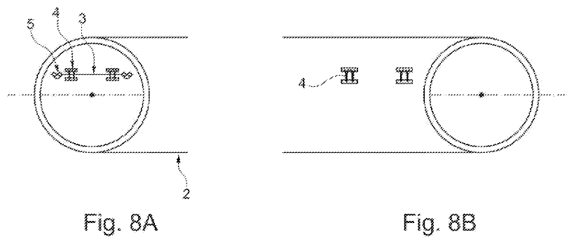

In FIGS. 8A and 8B, one can see a flexible elongated member in both internal (8A) and external (8B) views. As to FIG. 8A, an actuating means such as a shape memory alloy (SMA) wire 3 in anchored to the internal surface of the flexible elongated member 2. Ligation 4 windings are in axial abutting contact with the knot 5 so as to prevent the SMA wire 3 from sliding once actuated. The FIG. 8B is the view from outside showing ligation 4 windings going through recesses for the attachment.

While various embodiments have been described and illustrated, the detailed description is not to be construed as being limited hereto. Various modifications can be made to the embodiments by those skilled in the art without departing from the true spirit and scope of the disclosure as defined by the claims.

### REFERENCES

1—steerable device
2—flexible axially elongated member
3, 30—actuating means
4—fastening means
5, 51, 52, 53, 54, 55, 56, 57—anti-return means

1-18. (canceled)

19. A steerable device configured to be advanced in the lumen of a tubular element, said device comprising:
a flexible axially elongated member having proximal and distal ends,
at least one actuating means arranged alongside the periphery of said elongated member,
at least one fastening means configured to fasten at least partially the at least one actuating means to the flexible elongated member distal end, said fastening means being in direct contact with the at least one actuating means,
at least one anti-return means configured to keep the at least one actuating means from sliding alongside the periphery of the flexible elongated member distal end,
wherein the at least one anti-return means and the at least one fastening means are in axial abutting contact so as to prevent the at least one actuating means from sliding once actuated.

20. The steerable device according to claim 19, wherein the at least one actuating means is made of a shape memory alloy such as NiTi alloy.

21. The steerable device according to claim 19, wherein the at least one actuating means comprises a spring or a wire.

22. The steerable device according to claim 19, wherein the at least one flexible axially elongated member is wire or blade shaped.

23. The steerable device according to claim 19, wherein the flexible axially elongated member has a cross-section profile under a form selected from star, circular, semicircular, square, rectangular, triangle, pyramidal or any combinations thereof.

24. The steerable device according to claim 19, wherein the fastening means is a ligation around the flexible elongated member.

25. The steerable device according to claim 19, wherein the anti-return means is a tube, either glue filled or crimped, the actuating means going through said tube.

26. The steerable device according to claim 19, further comprising a second anti-return means made integral with the flexible elongated member so as to maintain the fastening means in place.

27. The steerable device according to claim 26, wherein the anti-return means is a recess built into the flexible elongated member and configured to host the at least one fastening means.

28. The steerable device according to claim 19, wherein the anti-return means is made integral with the actuating means.

29. The steerable device according to claim 28, wherein the at least one anti-return means is a encapsulating at least partially the at least one fastening means or said loop being an abutment for the at least one fastening means.

30. The steerable device according to claim 28, wherein the at least one anti-return means is a knot, a weld or any local reinforcement located at the at least one actuating means distal end.

31. The steerable device according to claim 28, wherein the at least one anti-return means is a groove built into the actuating means and configured to host the at least one fastening means.

32. The steerable device according to claim 31, wherein the groove is obtained by indentation.

33. The steerable device according to claim 19, wherein the at least one actuating means is inside an electrically isolating material such as a tube or a coating.

34. The steerable device according to claim 19, wherein the at least one actuating means is arranged alongside the external periphery of the elongated member.

35. An assembly method for steerable device according to claim 19, comprising the steps of:
providing a flexible axially elongated member having proximal and distal ends,
arranging at least one actuating means alongside the periphery of said elongated member,
providing at least one anti-return means configured to keep the at least one actuating means from sliding alongside the periphery of the flexible elongated member distal end,
fastening the at least one actuating means to the flexible elongated member distal end with at least one fastening means,
wherein the at least one anti-return means and the at least one fastening means are in axial abutting contact so as to prevent the at least one actuating means from sliding once actuated.

36. The assembly method according to claim 35, wherein the at least one actuating means is a wire, the at least one fastening means is a ligation and the at least one anti-return means is obtained by increasing the contact pressure between the wire and the ligation, with said ligation going alternatively up and under the wire so as to ligate longitudinally partial parts of the wire with the flexible elongated member.
